(19) United States
(12) Patent Application Publication
(10) Pub. No.: US 2016/0265057 A1
(43) Pub. Date: Sep. 15, 2016

(54) MARKERS FOR AMYOTROPHIC LATERAL SCLEROSIS (ALS) AND PRESYMPTOMATIC ALZHEIMER'S DISEASE (PSAD)

(71) Applicant: INSTITUTE FOR SYSTEMS BIOLOGY, Seattle, WA (US)

(72) Inventors: Jennifer Joy SMITH, Seattle, WA (US); Samuel Anthony DANZIGER, Seattle, WA (US); John David AITCHISON, Seattle, WA (US); Leslie Rae MILLER, Seattle, WA (US)

(73) Assignee: Institute for Systems Biology, Seattle, WA (US)

(21) Appl. No.: 15/025,207

(22) PCT Filed: Sep. 25, 2014
(86) PCT No.: PCT/US14/57530

§ 371 Date: Mar. 25, 2016

Related U.S. Application Data
(60) Provisional application No. 61/882,547, filed on Sep. 25, 2013.

(51) Int. Cl. C12Q 1/68

(52) U.S. Cl. CPC C12Q 1/6883; C12Q 2600/158

(57) ABSTRACT

Methods to detect amyotrophic lateral sclerosis (ALS) or presymptomatic Alzheimer's disease (PSAD) using an indicator cell assay platform (iCAP) in a test subject are described. Specifically, the disclosure provides a method comprising contacting a biological fluid of said test subject with indicator cells and assessing said indicator cells for the level of expression of an exon of CKIgamma2 that encodes the C-terminal palmitoylated region of said CKIgamma2, to determine the probability that a test subject is afflicted with amyotrophic lateral sclerosis (ALS). Further disclosed are methods of using indicator cells that are pan neuronal populations of glutamatergic (and/or GABAergic) neurons to determine the probability of the presence of presymptomatic or symptomatic Alzheimer's disease (PSAD) in a test subject.

### TECHNICAL FIELD

The invention is in the field of finding diagnostic assays for serious illnesses. In particular, it concerns a new marker that can be useful in diagnosing ALS and a method to detect ALS and PSAD.

### BACKGROUND ART

More than 5 million people in the US are currently living with AD. There is currently no cure or good treatment for AD, but early detection and management of the disease leads to reduced treatment cost and higher quality of life. Treatment of patients who are presymptomatic or have mild cognitive impairment (MCI), a condition that precedes the dementia characteristic of AD, can result in at least measured success. Use of therapeutics with a focus on treating presymptomatic AD (PSAD) is consistent with the fact that irreversible neuronal damage is detectible years to decades before onset of MCI. There is a critical need for reliable, low-cost non-invasive biomarkers of PSAD (for both early detection in the clinic and for drug efficacy testing by pharmaceutical companies); however, existing assays for direct detection of PSAD from serum remain unreliable despite many years of investigation.

Another problematic neurodegenerative disease is amyotrophic lateral sclerosis (ALS). ALS is extremely debilitating and can lead to weakness, paralysis and, ultimately, death. It is also known as Lou Gehrig's disease. The current state of diagnosis is complex and there are no known markers that are reliable for providing a useful diagnosis.

It is known that defects in the gene encoding TDP-43 can lead to ALS, and that misfolded TDP-43 is a major constituent in protein aggregates in many patients with ALS regardless of whether a mutation exists in this gene. (TAR DNA-binding protein 43 (TDP-43) is a transactive response DNA-binding protein with a molecular weight of 43 kD. It is a cellular protein which in humans is encoded by the TARDBP gene.) It is also known that TDP-43 aggregation is at first localized, but then spreads to neighboring unaffected neurons leading to more severe and widespread symptoms. One approach to disease progression is to stop the spread of protein aggregation that is transmitted from one cell to another, but the mechanism of spreading is not understood. One potential adjunct to such spreading is through a signaling molecule called casein kinase 1 gamma 2 (CK1γ2). It is changes in this protein that are the aspect of the present invention.

During ALS progression, there is an ordered spread of weakness and loss of motor control from point of onset to other regions in a spatiotemporal manner, suggesting the existence of soluble factors that can spread disease between cells. Consistent with this, in vitro models of ALS show that serum or cerebral spinal fluid from patients with ALS result in increased neuronal death. In addition, glial cells can spread toxicity to motor neurons in mice and in cell culture. These data demonstrate that ALS pathology can be spread from serum to cells, so that exposing cultured cells to serum is indicated as a method to identify and characterize cellular responses to signals of disease. As noted above, a proposed mechanism for the spread of disease to unaffected cells is the transfer of misfolded proteins from one cell to another, and conversion of normally folded proteins in the new cell into the aberrant conformation by a prion-like mechanism (Polymenidou M., et al., Cell (1997) 147:498-508). Misfolded proteins in ALS patients include SOD1, TDP-43 and FUS, and there is evidence for SOD1 acting as a template in this way, but evidence for the other proteins is lacking. Data showing that motor neuron toxicity in one system was mediated through glial SOD1 synthesis, suggests that ALS can spread from one cell to another in a SOD1 dependent manner and that prion-like spreading is a plausible explanation. However detection of toxicity transferred from human astrocytes to mouse motor neurons suggests the existence of a second mechanism (as human SOD1 is not a substrate that can seed mouse SOD1 aggregation. The present invention, in one aspect, concerns a novel second mechanism of ALS transmission between cells that is distinct from the prion model.

In relation to the foregoing, a hyper-phosphorylated, ubiquitinated and cleaved form of the TDP-43 (known as pathological TDP-43) is a major disease protein in ALS. Hyperphosphorylated TDP-43 is a major component of intranuclear and cytoplasmic inclusions deposited in brains of patients with ALS, which colocalize with stress granules. There are data in the art that suggest that a CK1 isoform may be involved in TDP-43 aggregation (Hasegawa, M., et al., Annals of Neurology (2008) 64:60-70; Inukai, Y., et al., FEBS Lett. (2008) 582:2899-2904; and Kametani, F., et al., Biochem/Biophys. Res. Comm. (2009) 382:405-409). These data include experiments with a truncated version of CK1δ with the C-terminal region deleted. This protein is called CK1 because it is missing the C-terminal region where the six CK1 isoforms (α, δ, ε, γ1, γ2 γ3) are most divergent. CK1 strongly phosphorylates TDP-43 in vitro, whereas phosphorylation by other kinases (CK2 or GSK3) is much weaker or was not detected. In addition, electrophoretic mobility shift of CK1-modified TDP-43 is similar to that of hyperphosphorylated TDP-43 associated with ALS in vitro.

Among 28 ALS-related mutations in TDP-43 (including pathologic mutations in familial cases and variants found in sporadic cases), all but one are in the C-terminal Gly-rich region (273-414) which is in the region hyperphosphorylated by CK1 (containing 18 of 29 mapped phosphorylation sites), and this region is required for TDP-43 aggregation and cellular toxicity in vivo. Together these data suggest a role for CK1 in TDP-43 phosphorylation and possibly aggregation, but they do not link CK1 to ALS. It is not known if CK1 activity on TDP-43 is activated by ALS progression, or which of the six isoforms is involved in TDP-43 phosphorylation. The invention, in one aspect, sheds light on these matters.

### DISCLOSURE OF THE INVENTION

In one aspect, the invention is directed to a method to determine the probability that a test subject is afflicted with amyotrophic lateral sclerosis (ALS) which method comprises contacting a biological fluid of said test subject with indicator cells and assessing said indicator cells for the level of expression of an exon of CK1γ2 that encodes the C-terminal palmitoylated region of said CK1γ2 whereby a diminished level of expression of this exon as compared to its expression level in said indicator cells when contacted with biological fluid of normal subjects indicates a high probability that said test subject is afflicted with ALS.

In another aspect, the invention is directed to a method to determine the probability of the presence of ALS in a test subject which method comprises using an indicator cell assay platform (iCAP) by contacting indicator cells that are motor neurons derived from stem cells with a biological fluid of said test subject and comparing the expression pattern in said indicator cells to that obtained when said cells are contacted with a biological fluid from normal subjects.

In another aspect, the invention is directed to a method to determine the probability of the presence of presymptomatic or symptomatic Alzheimer's disease (PSAD) in a test subject which method comprises using an indicator cell assay platform (iCAP) by contacting indicator cells that are pan neuronal populations of glutamatergic (and GABAergic) neurons with biological fluid of said test subject and comparing the expression pattern in said indicator cells to that obtained when said cells are contacted with biological fluid from normal subjects.

The platform iCAP is subject to a number of assay formats, but typically, the assays for expression in indicator cells are conducted by extracting mRNA, optionally obtaining corresponding cDNA, and then assessing the levels of the mRNA and/or cDNA using complementary probes thereto. Expression levels of specific genes are particularly useful in all of these determinations.

### BRIEF DESCRIPTION OF THE DRAWINGS

FIG. 1 shows differential splicing of CK1γ2 gene in response to ALS serum versus normal serum. Average log 2 intensities of each probe across the entire CK1γ2 transcript are shown (including data from 11 and 12 experiments with serum from presymptomatic ALS and normal mice, respectively). For most probes/exons, expression in response to ALS serum or normal serum is similar. One putative differentially spliced exon (probe 15) is circled.

FIG. 2 shows differential abundance of the CK1γ2 probe in the disease signature (correspond to probe 15 in FIG. 1) in response to disease and normal serum. Probe intensities (calculated using FIRMA software (Purdom, E. et al., Bioinformatics (2008) 24:1707-1714)) are relative to intensities of other probes in the same gene on the same array. Box plots show median log 2 (expected/actual intensity) for the probe across 20 and 21 experiments with serum from presymptomatic ALS and normal mice, respectively, along with boxes depicting the first and third quartiles. Student's t-test p-value comparing data from normal and disease samples is 0.015.

FIG. 3 shows the differentially expressed exon encodes the extreme C-terminus of CK1γ2. Protein sequence of CK1γ2 is shown with amino acids colored according to the exons encoding them. Alternate exons (light) and amino acids encoded across splice sites (bold and italicized) are shown. The position of the Affymetrix® probe representing the differentially expressed exon is indicated by asterisks. The position of the predicted palmitoylation domain is underlined.

FIG. 4 shows boxplots of median accuracies of ALS classifiers with various training subsets when tested on an independent blind dataset of 24 samples. Boxplots of Matthews correlation coefficients are also shown. Each classifier was composed of ˜60 differentially expressed gene pathways (of ˜10, 000 total pathways).

FIG. 5 is a graph showing the number of paired disease/normal assays needed for PSAD as a function of the number of significantly differentially expressed exons in the PSAD signature.

### MODES OF CARRYING OUT THE INVENTION

U.S. patent publication US2012/0245048, the contents of which are incorporated herein by reference, describes an assay designed to detect the presence of ALS by assessing the biological fluid of a test subject for markers that result from treating said biological fluid with spinal motor neurons derived from HGB3 embryonic stem cells. Using this assay, it is found that, as shown in the examples below, the CK1γ2 transcript showed reduced expression of the exon encoding the small C-terminal regulatory region of CK1γ2 which is both palmitoylated and phosphorylated.

Palmitoylation of CK1γ (a closely related Xenopus isoform of CK1γ2) facilitates targeting and tethering of the kinase to the plasma membrane where it is localized under normal conditions. Failure of the mouse exon to be fully expressed should therefore results in a reduction in the amount of protein that is tethered to the plasma membrane and increases the cytoplasmic pool (as has been observed for CK1γ truncations in Xenopus). These data indicate that in the cytoplasm, the CK1γ2 can propagate ALS pathology by phosphorylation of TDP-43 (as has been shown for CK1 in vitro). As noted above, hyperphosphorylation of TDP-43 is characteristic of ALS. Thus, the underexpression of this exon results in a known factor that propagates ALS. One method for ascertaining the expression of the exon is to assess the localization CK1γ2 in cytoplasm of indicator cells.

While use of motor neurons as indicator (responder) cells is contraindicated in the case of Alzheimer's diagnosis, the general approach for detecting ALS is a good surrogate for AD or PSAD since both are neurodegenerative diseases with common underlying pathologies; both are caused by late onset protein misfolding and toxic aggregation, and involve common cellular processes including the ubiquitin-proteasome, programmed cell death, ROS overproduction, and dysfunctional mitochondria and axonal transport (Jellinger, K. A., J. Cell. Mol. Med (2010) 14:457-487; Jellinger, K. A., J. Neural Transm. (2009) 116:1111-1162); Federico, A. et al., J Neurol. Sci (2012) 322:254-262).

A common emphasis on exons results in a determination of splicing as a differential in disease states as compared to normals. Splicing effects about 80% of human genes and aberrant alternative splicing is already linked to neurodegenerative disease and related cellular dysfunctions including proteasome inhibition, and oxidative stress. Splice variants specific for AD and Parkinson's disease have been identified in blood (Potashkin, J. A., et al., PLoS One (2012) 7:e43595 and Fehlbaum-Beurdeley, P., et al., J. Alzheimer's Assoc. (2010) 6:25-38). Splicing can be identified by within-sample comparisons thus diminishing technical error due to between-sample comparisons.

An emphasis on pathways (gene sets) results in determination of gene set enrichment as a differential in disease state as compared to normals. This approach measures expression of gene sets (genes involved in a common cellular pathway or sharing another annotation) instead of individual genes, effectively reducing the number of features considered and identifying statistically significant differential expression of some genes that would otherwise go unnoticed due to noise in the measurement (Subramanian, A., et al., PNAS (2005) 102:15545-15550).

Using pan neuronal glutamatergic (mixed with GABAergic) cells as responders to compare early stage AD plasma samples (post-MCI) to those from cognitively normal subjects (4 replicates of each) (for exon level analysis without disease classification), a t-test was performed (without multiple testing correction) and 2,537 exons were significantly differentially spliced (p-value <0.05). A power calculation was performed suggesting that a significant differential response signature of ˜1000 exons can be generated using data from 20 paired disease/normal experiments.

The assays of the invention can use blood, including serum, and cerebrospinal fluid (CSF) samples which could be run concomitantly. In some assays, the responder cells are grown for 5 days to a steady level of responsiveness and exposed to CSF or serum or other bodily fluid for 24 hours. Transcriptome profiles can be analyzed using Affymetrix® human exon assays.

For using an iCAP to classify the disease state of new subjects, differential gene expression profiles can be used to train a disease classifier to classify new subjects based on their expression profile in the same cell based assay. This can involve first selecting a subset of features (genes, gene sets or exons) that are differentially expressed in the iCAP signatures of disease versus normal subjects using a machine-learning feature selection tool like mProbes (Huynh-Thu, V. A. et al., Bioinformatics (2012) 28:1766-1774), and next training and testing a disease classifier using machine-learning approaches like support vector machines (SVM; Furey, T. S. et al., Bioinformatics (2000) 16:906-914; Brown, M. P. et al., PNAS (2000) 97:262-267).

While a wide variety of assay formats for expression is available, in the examples below, expression levels are determined by obtaining mRNA from the indicator cells, optionally preparing complementary DNA corresponding to the mRNA extracted and assessing the mRNA and/or cDNA for binding to complementary probes. It is possible to assess multiple mRNA and/or cDNA levels at once using arrays of probes, many of which are commercially available.

Further, in the examples below, in addition to the specific detection of expression of the C-terminal palmitoylated region of CK1γ2 for ALS, an overall expression pattern can be obtained for diagnosis both of ALS and symptomatic and presymptomatic AD. In the examples below, specific genes that are over- or under-expressed in the presence of these abnormal conditions when biological fluid from a test subject is contacted with the indicator cells are disclosed. In the case of ALS, murine subjects and indicator cells were used and the genes represented in the array represent murine genes. The method is equally applicable to the ortholog genes in humans and other species. Thus, the methods of the claims are applicable to test samples from any subject susceptible to ALS including mammals in general and especially humans. The illustrative work with regard to AD in Example 2, however, specifies human genes.

The number of genes whose expression levels are to be tested is subject to the judgment of the practitioner. As few as two or as many as 50 or more may be determined simultaneously to obtain a pattern. Thus, one could choose to detect expression levels of, for example, 5, 10, 20, 30, 40, 50 or 100 genes. In the case of ALS, all of the more than 400 specified genes may be assessed. These ranges are intended to include all intervening integers rather than taking up space to articulate each integer specifically, the inclusion of intermediate values is simply referred to herein.

The following examples are intended to illustrate but not limit the invention.

### EXAMPLE 1

### Detection of an ALS Marker

The ALS signature in serum of mice developing ALS was determined using motor neurons as detector cells as described in US2012/0245048. Motor neurons have been shown to be targeted by the disease in a non-small cell autonomous manner (Nagai, M, et al., Nature Neuroscience (2007) 10:615-622), and therefore are responsive to disease-specific signatures in serum.

In one experiment, as set forth in the above-mentioned publication, disease serum was taken from 5 transgenic ALS susceptible mice (SOD1; G93A) at 9 weeks of age and control serum was taken from 5 non-carrier mice of the same age from the same colony.

Spinal motor neurons (MNs) were derived from HGB3 embryonic stem cells expressing a fluorescently labeled motor neuron marker (HB9-eGFP) by a method previously described (Wichterle, H., et al., Cell (2002) 110:385-397) as described below. Unless otherwise specified, growth of ES cells was in differentiation medium (consisting of equal parts Advanced™ DMEM/F12 (Invitrogen) and Neurobasal™ medium (Invitrogen) supplemented with penicillin/streptomycin, 2 mM L-Glutamine, 0.1 mM 2-mercaptoethanol, and 10% KnockOut™ serum replacement (Invitrogen)). ES cells were plated at ˜10⁵ cells per mL and grown in aggregate culture for 2 days to form embryoid bodies (EBs) in a 10 cm² dish. EBs were split 1:4 into four 10 cm² dishes and exposed to 1 μM each retinoic acid and sonic hedgehog agonist (Hh-Ag1.3, Curis, Inc.) for two days, to caudalize spinal character and ventralize into MN progenitors, respectively. Medium was changed and EBs were grown for an additional 3 days in differentiation medium to generate MNs. Two dishes of EBs were pooled, washed with PBS and resuspended in 1 mL of differentiation medium. 100 μL of these EBs were inoculated in each of 10 wells of a 3.8 cm² 12-well dish. EBs were incubated for 24 h in 2 mL differentiation medium containing either 5% serum from 9 week-old ALS susceptible mice or 5% serum from normal mice. Each experiment (disease or control) was done five times with serum from five different mice.

RNA was isolated using TRIzol® reagent, and cDNA was synthesized from polyA RNA, labeled and hybridized to Affymetrix® GeneChip® mouse exon arrays according to manufacturer's recommendations.

Probe intensities for ten experiments (five replicates each of control and disease serum) were normalized together and data from probes representing a continuous stretch of putatively transcribed genomic sequence were merged into probe sets (using RMA algorithm of the Affymetrix® Expression Console software). Two filters were applied to exclude probe sets that did not meet the criteria below: 1. Probe sets map to the genome and thus levels are annotated as “core”, “full”, “free” or “extended” by Affymetrix®. 2. Probe sets have high confidence of detection over background in at least 5 of the 10 experiments (P<0.001 determined using the DABG algorithm of the software). After application of these two filters, the data set consisted of 135,181 probe sets.

Probe-level expression values were analyzed for significant differential expression between cells exposed to control serum and those exposed to disease serum using Significance Analysis for Microarrays (SAM) of MeV component of TM4 microarray software (by running a two-class paired analysis using default parameters and the 32 possible unique permutations of the data to calculate the statistic). This analysis generated an ALS disease signature consisting of 441 probe sets that significantly increased in expression in response to disease serum compared to normal serum with q-values and false discovery rates <15%.

The high level of resolution of the above exon arrays was accessed in analysis of differential splicing of mRNA in response to pre-symptomatic ALS mouse serum (versus normal mouse serum) using FIRMA software (Purdom, E., et al., Bioinformatics (2008) 24:1707-1714. The comparison of genes together within the same sample makes the tests invariant to all forms of data normalization that do not affect within-sample quantification. For this analysis, additional data were generated resulting in a total of 41 datasets (including responses to serum from presymptomatic ALS mice (N=20) and age-matched normal mice (N=21)). Next, splice variants were identified and used to find disease-specific differentially expressed exons. Next, exons were ranked by magnitude of differential splicing and disease classification was performed in two steps: 1) Ranked exons were used to build and train an ensemble of classifiers using only half of the samples (11 ALS and 12 normal). The ensemble predicted the remaining 18 independent samples, revealing the classifier accuracy as 82% (p-value <0.001). 2) The top 100 ranked exons from 1) were used to train and test a new classifier using all of the samples. Leave-one-out cross validation predicts classifier accuracy of 78% (p-value <0.0001).

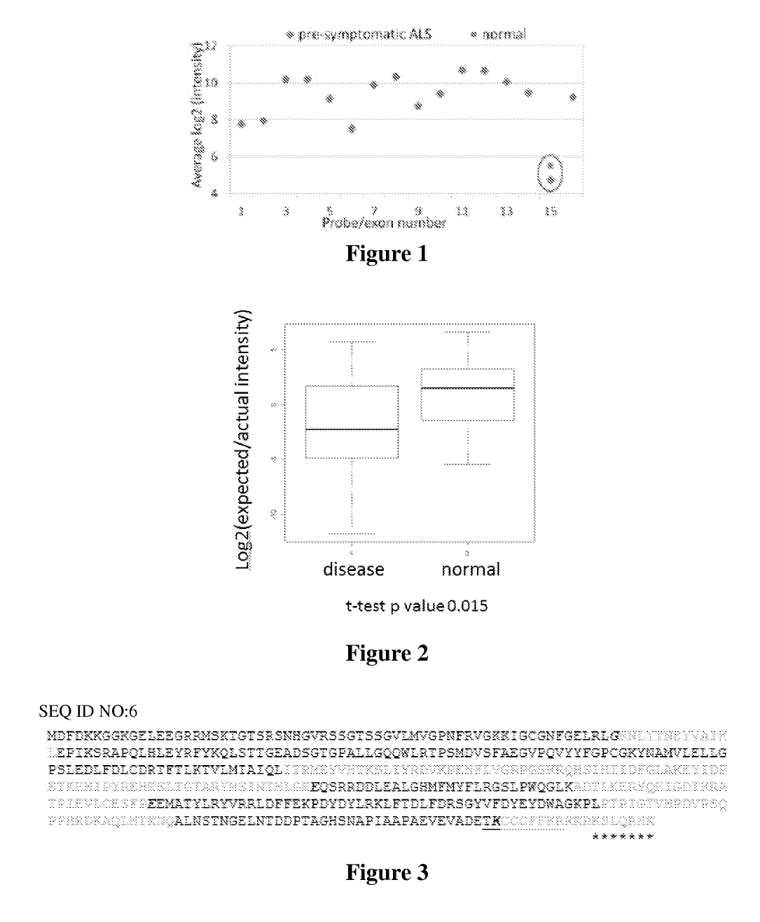

CK1γ2, the top ranked significantly differentially spliced genes in the disease signature, was further characterized to predict its involvement in a cellular response to presymptomatic ALS serum. Differential splicing was analyzed, whereby average intensities for all probe sets within the putative CK1γ2 transcript (supported by RefSeq and full-length mRNA GenBank records) are shown in FIG. 1. Despite the existence of 6 closely related CK1 isoforms, all probe sets analyzed are unique (perfectly match only one sequence in the putatively transcribed array content) (affymetrix.com). Most probe targets tested appear to be of similar abundance in disease versus normal samples (i.e., have similar detected intensities), but one exon (probe 15) is of lower abundance in response to pre-symptomatic ALS serum versus normal serum. These data suggest differential splicing of CK1γ2 in response to presymptomatic ALS serum. Importantly, these results have been validated by repeating the experiment using serum samples from independent mice that were not part of the previous analysis and the same results were obtained (data not shown). To further support differential expression of the CK1γ2 exon in the disease signature, the distributions of expression values for the probe were analyzed (FIG. 2). The distributions for disease and normal samples are significantly different from each other (t-test p-value=0.015). The differentially expressed probe (SEQ ID NO:5) is in an exon at the extreme 3′ end of the open reading frame. The exon encodes the extreme C-terminus of the encoded protein (containing 18 of 442 amino acids) (last exon shown in FIG. 3). Although the splicing is toward the end of the gene, it is not at the end of the transcript, and the last exon in the transcript is not differentially expressed; therefore, the observation is not likely to be due to an artifact of transcript degradation. The putative differentially regulated exon has a predicted palmitoylation domain (underlined in FIG. 3) for appending a fatty acid to a protein to stabilize membrane binding which has been shown to be necessary for tethering Xenopus CK1γ, a closely related isoform to the plasma membrane. Additionally, CK1γ2 is phosphorylated and the only phosphorylation site seen by 8 independent MS experiments is the serine in the differentially expressed exon (S437) (phosphosite.org). Thus, exposure of motor neurons to ALS serum results in differential splicing that likely results in relocalization of CK1γ2 from the plasma membrane to the cytoplasm.

The sequences used in the foregoing assay are as follows:

Sequence of the differentially expressed probe on
the Affymetrix ® microarray (Mouse Exon 1.0 ST):
(SEQ ID NO: 5)
AAATCGCTGCAGCGACATAAG
Sequence of Mouse CK1γ2 exon containing the probe:
(SEQ ID NO: 4)
AAGTGCTGCTGCTTCTTCAAGAGGAGAAAGAGAAAATCGCTGCAGCGACA
TAAGTGA
Encoded mouse peptide sequence:
(SEQ ID NO: 3)
KCCCFFKRRKRKSLQRHK
Human Ensembl gene identifier:
ENSG00000133275 (Csnk1g2)
Sequence of corresponding human exon CK1γ2 exon:
(SEQ ID NO: 2)
AAATGCTGCTGTTTCTTCAAGAGGAGAAAGAGAAAATCGCTGCAGCGACA
CAAGTGA
Corresponding human peptide sequence:
(SEQ ID NO: 1)
KCCCFFKRRKRKSLQRHK

Next an iCAP-based classifier was developed for ALS detection from serum using the same cell-based assay except with analysis of gene-level and exon-level expression data. For this analysis, additional data were generated resulting in a total of 47 datasets (including data using serum from presymptomatic ALS mice (N=23) and age-matched normal mice (N=24)).

Data were merged and two filters were applied to exclude probe sets that did not map to a gene, and probe sets that did not have high confidence of detection over background in at least one experiment (P<0.01 determined using the DABG algorithm of the software).

All data were co-normalized (Purdom, E. et al., Bioinformatics (2008) 24:1707-1714), and half of the data (12 of control class and 11 of disease class) were used to build a disease classifier. To do this, three feature types were analyzed for significant differential enrichment between the classes including splice variants (Purdom, E., et al., Bioinformatics (2008) 24:1707-1714; Irizarry, R. A., et al., Nucleic Acids Res. (2003) 31:e15; Irizarry, R. A., et al., Biostatistics (2003) 4:249-264), genes and pathways (Efron, B., et al., The Annals of Applied Statistics (2007) 1:107-129). Pathways are sets of genes share a common annotation including those from GO, KEGG and REACTOME, and were used as features in attempt to capture complex interactions between variables.

Next, features were selected by ranking (based on magnitude and significance scores) and using mProbes, a machine-learning feature selection tool that uses artificially generated random features to generate a noise model (Huynh-Thu, V. A. et al., Bioinformatics (2012) 28:1766-1774), to select top features that rise above the noise for classification (FDR <100% or other metrics).

Sets of selected features were used to build and train disease classifiers using Support Vector Machines (SVM) with polynomial kernels (an approach that performs well with the large number of features of gene expression datasets) (Furey, T. S., et al., Bioinformatics (2000) 16:906-914; Brown, M. P., et al., PNAS (2000) 97:262-267), or an ensemble of this SVM with random forest (Breiman, L., Machine Learning (2001) 45:5-32), evolutionary tree and naïve Bayes classifiers. All classifiers were tested by predicting the remaining 24 independent blind samples (12 of each class).

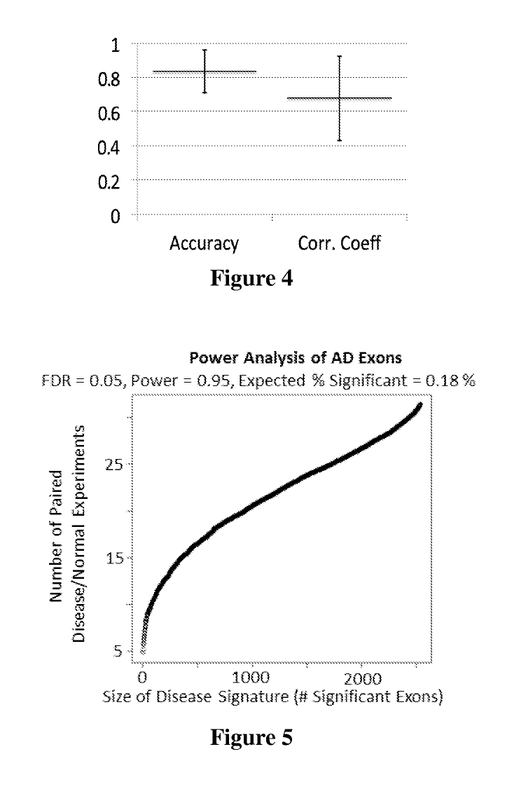

Top classifier performance was observed for iterations using pathway features (absolute GSA scores ≧1) and SVM classification (accuracies of 83-96%). Iterations using pathway features with other classifiers were not as accurate, but performed significantly better than random. To evaluate classifier robustness, one method was selected (SVM classification using mProbes-selected pathway features (absolute GSA scores ≧1 and FDR<100%)) and the analysis was repeated with 24 subsets of the training data (each with one feature removed). Each classifier was made up ˜60 pathway features (representing ˜430 genes). The classifiers performed well with a top classifier accuracy of 96% and correlation coefficient of 0.92 (FIG. 4).

Significantly differentially expressed features of the iCAP reflect known aspects of ALS: 1) Gene pathways include the ER stress response mediated by PERK (and transcription factors (TFs), ATF4 and CHOP) (Han, J., et al., Nature Cell Biology (2013) 15:481-490), an early pathological event in ALS (Saxena, S. and Caroni, P., Neuron (2011) 71:35-48) and 2) Gene list includes ATF4 and CHOP (Ddit3) and is enriched for their known targets (Han, J., et al., Nature Cell Biology (2013) 15:481-490). Genes are also significantly enriched for those specifically expressed in microdissected neurons from presymptomatic SOD1 ALS mice (Lobsiger, et al., PNAS (2007) 104:7319-7326; Ferraiuolo, L., et al., J. Neuroscience (2007) 27:9201-9219; Perrin, F. E., et al., Human molecular genetics (2005) 14:3309-3320).

These data establish feasibility of developing a robust iCAP-based classifier for detection of presymptomatic ALS using human serum. In addition to disease classification, the assay may have other utility; significantly differentially expressed features of the iCAP are enriched for genes and processes that have been implicated in ALS, suggesting that the assay may also have utility for understanding disease mechanism and identifying candidate therapeutic targets.

The genes in the pathways used to train the classifier with the top performance (SVM classification of mProbes-selected pathway features (absolute GSA≧1 and FDR<100%) are listed below:

| | | | | |
|---|---|---|---|---|
| 1) UBE2A | 2) UBE2B | 3) RNF8 | 4) UBR2 | 5) MARS |
| 6) BCAR1 | 7) SPG21 | 8) SLA2 | 9) OAT | 10) PYCR1 |
| 11) ALDH18A1 | 12) PYCR2 | 13) PYCRL | 14) GARS | 15) SMAD1 |
| 16) POLB | 17) POLG2 | 18) TARS | 19) TARS2 | 20) TARSL2 |
| 21) MTHFD1 | 22) MTHFD2 | 23) MTHFD1L | 24) MTHFD2L | 25) B4GALT1 |
| 26) B4GALT3 | 27) B4GALT2 | 28) WDFY3 | 29) SLC3A2 | 30) SLC8A2 |
| 31) SLC8A1 | 32) SLC8A3 | 33) INPP5A | 34) INPP5B | 35) INPP5J |
| 36) INPP5K | 37) NAT1 | 38) SLC1A4 | 39) SLC1A5 | 40) SLC38A3 |
| 41) SLC38A7 | 42) MTHFS | 43) MTHFSD | 44) MTHFR | 45) SHMT1 |
| 46) SHMT2 | 47) FTCD | 48) ALDH1L1 | 49) MTFMT | 50) ALDH1L2 |
| 51) DHFR | 52) GART | 53) AMT | 54) MTR | 55) ATIC |
| 56) TYMS | 57) SLC36A4 | 58) SLC36A2 | 59) CLN8 | 60) GAA |
| 61) GCH1 | 62) GLRA1 | 63) HEXA | 64) SCN1A | 65) TCF15 |
| 66) CNTNAP1 | 67) SLC7A1 | 68) SLC7A3 | 69) SLC7A5 | 70) SLC7A11 |
| 71) PIPDX | 72) FGF2 | 73) SMAD3 | 74) SERPINE1 | 75) CASK |
| 76) PTCH1 | 77) PTCH2 | 78) HHIP | 79) GPT | 80) GPT2 |
| 81) ASNS | 82) ATF3 | 83) CCL2 | 84) CEBPZ | 85) DDIT3 |
| 86) HERPUD1 | 87) IGFBP1 | 88) AARS | 89) IARS | 90) VARS |
| 91) VARS2 | 92) LARS2 | 93) LARS | 94) IARS2 | 95) IL18 |
| 96) PDE2A | 97) PDE3A | 98) VEGFA | 99) FGFBP3 | 100) PGD |
| 101) PHGDH | 102) PSAT1 | 103) FOXC1 | 104) HEXB | 105) CLN6 |
| 106) GPLD1 | 107) MEF2C | 108) PPARGC1B | 109) FGFR3 | 110) IHH |
| 111) DDR2 | 112) TKT | 113) FLT3 | 114) HELLS | 115) HPRT |
| 116) IMPDH1 | 117) IMPDH2 | 118) RAD23A | 119) RAD23B | 120) WNT10B |
| 121) UBQLN4 | 122) DNASE1L1 | 123) DNASE1L2 | 124) DNASE1L3 | |
| 125) TATDN21 | 26) TATDN3 | 127) ROS1 | 128) AGPAT9 | 129) PGK1 |
| 130) PGK2 | 131) FAS | 132) FASN | 133) NDUFAB1 | 134) HK1 |
| 135) KCNA4 | 136) KCNJ11 | 137) PKLR | 138) PKM | 139) PDXK |
| 140) HDAC4 | 141) PHF2 | 142) KDM1A | 143) KDM4C | 144) PHF8 |
| 145) JHDM1D | 146) EHMT2 | 147) SMYD2 | 148) EHMT1 | 149) SETD7 |
| 150) SETD3 | 151) CNN2 | 152) PRTN3 | 153) TGFB1 | 154) ADIPOQ |
| 155) GNB2L1 | 156) EIF2AK3 | 157) HSPA5 | 158) EIF2A | 159) EIF2S1 |
| 160) ATF4 | 161) DDR1 | 162) GLI2 | 163) LHX1 | 164) RELN |
| 165) VLDLR | 166) ARNT | 167) EPAS1 | 168) HLF | 169) HIF1A |
| 170) HMOX1 | 171) SIN3A | 172) FOXC2 | 173) PTGS2 | 174) HDAC7 |
| 175) SRPX2 | 176) ITPR1 | 177) ITPR2 | 178) ITPR3 | 179) CYTH3 |
| 180) BLM | 181) MYC | 182) TXNIP | 183) NUMA1 | 184) PRM1 |
| 185) PRM2 | 186) ATXN7 | 187) SYNE1 | 188) HSF4 | 189) KDM3A |
| 190) ABCA1 | 191) MTTP | 192) ATG7 | 193) ATG10 | 194) PPP1R12A |
| 195) SIP1 | 196) ZEB2 | 197) BMP2K | 198) SBF2 | 199) PDK1 |
| 200) PDK2 | 201) PDK3 | 202) PDK4 | 203) BCKDK | 204) KCNN1 |
| 205) KCNN2 | 206) KCNN3 | 207) KCNN4 | 208) EEF1E1 | 209) EPRS |
| 210) QARS | 211) AIMP2 | 212) AIMP1 | 213) RARS | 214) DARS |
| 215) KARS | 216) NARS | 217) CARS | 218) HARS | 219) FARSA |
| 220) FARSB | 221) PPA1 | 222) SARS | 223) YARS | 224) DHH |
| 225) CSRP2BP | 226) B4GALT4 | 227) ORC1 | 228) ORC2 | 229) SLC7A2 |
| 230) SLC25A15 | 231) SLC25A2 | 232) SNCA | 233) MFN2 | 234) TIMM50 |
| 235) CDH1 | 236) FLNA | 237) DDX58 | 238) EAF2 | 239) DMAP1 |
| 240) MAVS | 241) TMEM173 | 242) CDK6 | 243) DRD1A | 244) GFAP |
| 245) GIF | 246) LAMB2 | 247) MT3 | 248) POU3F2 | 249) EIF2B5 |
| 250) LAMC3 | 251) SUV39H1 | 252) BAZ2A | 253) RRP8 | 254) SIRT1 |
| 255) FCER1G | 256) HRG | 257) SYK | 258) TEC | 259) GANC |
| 260) MGA | 261) MGAM | 262) DECR1 | 263) ECSIT | 264) MIOX |
| 265) WDR93 | 266) CHRNA1 | 267) CHRND | 268) VPS54 | 269) TSHZ3 |
| 270) DLAT | 271) MLYCD | 272) ACSS1 | 273) FGFR4 | 274) FIGF |
| 275) CCL5 | 276) VEGFB | 277) VEGFC | 278) FBP1 | 279) PPARA |
| 280) IER3 | 281) DDIT4 | 282) NCKAP1L | 283) LCK | 284) STAT5A |
| 285) STAT5B | 286) GIMAP5 | 287) CREBBP | 288) TSC22D3 | 289) BHLHE40 |
| 290) STRA13 | 291) BHLHE41 | 292) SLC1A1 | 293) SLC1A2 | 294) SLC1A6 |
| 295) SLC1A7 | 296) TNFSF10 | 297) TNFRSF10B | 298) FADD | 299) CASP8 |
| 300) ACVR1 | 301) EFNA1 | 302) SOX4 | 303) TWIST1 | 304) IL2 |
| 305) IL21 | 306) GTPBP1 | 307) CARHSP1 | 308) EXOSC3 | 309) DIS3L |
| 310) RS1 | 311) ARL6IP5 | 312) TRAT1 | 313) YRDC | 314) PARP1 |
| 315) PNKP | 316) MRPS35 | 317) MRPS26 | 318) MRPS11 | 319) MRPS9 |
| 320) SLC7A7 | 321) SLC7A15 | 322) SLC7A8 | 323) SLC7A4 | 324) SLC7A9 |
| 325) SLC7A10 | 326) SLC7A6 | 327) SLC7A6OS | 328) SLC7A12 | 329) SLC7A13 |
| 330) SLC7A14 | 331) DNASE1 | 332) DNASE2A | 333) SOX11 | 334) NKX2.5 |
| 335) NOTCH1 | 336) HDAC5 | 337) MYOCD | 338) DNA2 | 339) MDP1 |
| 340) POLG | 341) RNH1 | 342) DNAJA3 | 343) RRM2B | 344) PEO1 |
| 345) RNASEH1 | 346) ENSA | 347) KCNJ12 | 348) KCNMB2 | 349) KCNV1 |
| 350) PDZD3 | 351) TNFRSF11B | 352) CALCA | 353) CD38 | 354) INPP5D |
| 355) P2RX7 | 356) TNFAIP3 | 357) CARTPT | 358) KDR | 359) PTPRJ |
| 360) SDC4 | 361) SFRP1 | 362) TEK | 363) TSC1 | 364) PPM1F |
| 365) AMBP | 366) BLVRA | 367) BLVRB | 368) HMOX2 | 369) SMAD4 |
| 370) TGFB2 | 371) NF1 | 372) POU3F1 | 373) SKI | 374) ARHGEF10 |
| 375) ADAM22 | 376) LGI4 | 377) TOP1 | 378) TOP3A | 379) TOP3B |
| 380) TOP1MT | 381) BMP4 | 382) FOXJ1 | 383) ZC3H8 | 384) NFKBID |
| 385) BCKDHA | 386) BCKDHB | 387) DBT | 388) NAT2 | 389) SAT1 |
| 390) LAT2 | 391) SLC43A1 | 392) SLC6A15 | 393) SLC38A1 | 394) SLC6A17 |
| 395) AGRP | 396) CNR1 | 397) HTR1A | 398) TACR3 | 399) QRFP |
| 400) MIF | 401) MC1R | 402) AKAP5 | 403) AKAP12 | 404) CCR4 |
| 405) PARN | 406) PAN2 | 407) CNOT6 | 408) CNOT6L | 409) PIM1 |
| 410) LONP1 | 411) CLPX | 412) CRBN | 413) LONRF3 | 414) LONP2 |
| 415) LONRF1 | 416) LONRF2 | 417) ADM | 418) HES1 | 419) RAMP2 |
| 420) HEY2 | 421) CCBL1 | 422) GLS | 423) GLUD1 | 424) GLUL |
| 425) GOT1 | 426) GOT2 | 427) PAH | 428) GLS2 | 429) CAD |
| 430) DFFA | 431) DFFB | 432) NME1 | | |

### EXAMPLE 2

### Alzheimer's Assay

A mix of iPSC-derived glutamatergic and GABAergic neurons (from Cellular Dynamics International) were plated in a 12-well dish (at 600,000 cells/well) and cultured for 5 days. Cells were then exposed to 5% plasma from 4 cognitively normal controls, and 4 patients with confirmed mild cognitive impairment (MCI) for 24 h and RNA was isolated and used for gene expression analysis using Affymetrix® human exon arrays (ST 1.0). The data were merged, normalized, and filtered to include only ˜207,000 of the ˜1.4 M exons on the array that were significantly detected above background (DABG <0.01) for either all of the normal or all of the early symptomatic AD (PSAD) experiments. A t-test was performed on individual exons (i.e., without multiple test correction) and revealed significant differential splicing of 2,537 exons (p-value <0.05) in response to early symptomatic AD versus normal plasma.

The exons in the disease signature correspond to 2,234 genes. Because AD pathogenesis is strongly linked to production and deposition of the beta amyloid peptide, these genes were analyzed for enrichment of the NCBI gene description term “amyloid beta” as a preliminary analysis of AD relatedness. The genes in the preliminary disease signature were significantly enriched for the term “amyloid beta” when compared to all expressed genes on the array (HGD p-value <0.05).

These data formed the basis of a power analysis to estimate the number of experiments needed to obtain significant differential gene splicing between normal and PSAD serum samples in the iCAP (using a t-test with an FDR threshold of 0.05 and a Beta of 0.05). The analysis estimated that performing 20 paired disease/normal experiments would yield a signature made up ˜1000 significantly differentially spliced exons (see FIG. 5).

To perform this analysis, the fraction of all transcripts that are expected to be significant from the preliminary AD analysis was calculated. The power.t.test.FDR function in the [R] ‘ssize’ (Warnes, G. R., et al., (2012) “ssize: Estimate Microarray Sample Size”. R package version 1.32.0) toolbox was used to get a false discovery rate (FDR) power analysis estimate for these 2,537 exons. The FDR threshold was set to 0.05, the power to 0.95, and the expected fraction of significant exons to ˜0.002 (i.e., 2,537/1,432,336) to calculate the total number of paired AD/normal experiments needed to reach statistical significance after FDR correction (Note: larger fractions, such as those that use 207,789 instead of 1,432,336 would result in smaller numbers of experiments). As shown the results range from 5 experiments (i.e., one additional AD and one additional normal experiment) for one exon to 32 experiments (i.e., 28 additional AD and 28 additional normal experiments) for all 2,537 exons.

Next, the iCAP was used to train and test a disease classifier for presymptomatic AD. To do this, the assay was repeated with plasma samples from three classes of patients: 1) pre-MCI (cognitively normal patients with AD biomarkers present in CSF), 2) MCI/early AD (patients with mild cognitive impairment (MCI) (Rosen, C., et al., Mol. Neurodegener (2013) 8:20) or early AD), and 3) healthy controls (cognitively normal patients with AD biomarkers not present in CSF).

The data for 15 samples of each class were merged and normalized (Purdom, E., et al., Bioinformatics (2008) 24:1707-1714). Three feature types were analyzed for significant differential enrichment between the classes including genes, splice variants, and pathways (as was done for the ALS iCAP described in Example 1).

Significant differential expression of pathways is reflected by gene set enrichment (GSE) scores calculated using GSEA algorithm (Efron, B. and Tibshirani, R., The Annals of Applied Statistics (2007) 1:107-129). GSE scores with absolute values greater than 1 were considered significantly differentially expressed. Of the total 9633 pathways, 368 were significantly differentially expressed for Pre-MCI versus normal samples and 526 were significantly differentially expressed for MCI/early AD versus normal samples. Comparison of these two pathway sets showed a statistically significant overlap of 205 pathways (hypergeometric distribution probability of 1×10E-177) and these pathways showed either increased or decreased expression in response to disease in both datasets. These data suggest that human blood will be a viable source of AD-specific factors that are detectable using the iCAP, and that data from later-stage patients can be used to build classifiers for early-stage AD.

The gene expression data were used to generate a preliminary disease classifier for AD. To do this, first pre-MCI and MCI/early AD disease samples (30 total) were grouped for comparison against normal samples (15 samples up-sampled to 30).

Next, the top differentially expressed genes between disease and normal samples were selected (from ˜20,000 genes) using three criteria: significance of differential gene expression (t-test p-value), magnitude of differential gene expression (fold change ratio), and significance of differential expression of pathways associated with each gene (pathways were genes sets selected using GSEA algorithm; Efron, B. and Tibshirani, R., The Annals of Applied Statistics (2007) 1:107-129).

Next, an approach was used to find the optimal number of features to build the classifier. This was done by generating various subsets of the top-ranked features, and selecting the smallest subset that maximized the number of informative features for classification (evaluated using a random forest feature selection tool of mProbes; Huynh-Thu, V. A. et al., Bioinformatics (2012) 28:1766-1774). Using this approach, a random forest classifier was trained using the top 500 features.

The classifier was validation against 20 new blind samples that were independent from the samples used to train the classifier. The blind predictive accuracy of the classifier was tested on various subsets of the top ranked genes. Including between 50 and 500 genes results in a classifier accuracy between 75-80%.

Top ranked 50 features used to build the AD iCAP classifier are listed below. APOE, a gene with variant that is the largest known genetic risk factor for late-onset sporadic Alzheimer's disease in several ethnic groups (Sadigh-Eteghad, S. et al., Neurosciences (Riyadh) (2012) 17:321-326), is ranked third.

| | | | |
|---|---|---|---|
| 1) MYLK2 | 2) TOMM20L | 3) APOE | 4) ZNF675 |
| 5) MYLK3 | 6) SULT2B1 | 7) GRIA2 | 8) LCAT |
| 9) GRIA4 | 10) IL18 | 11) OSR2 | 12) ZNF525 |
| 13) IL4 | 14) TAS2R50 | 15) GHRL | 16) DBP |
| 17) IHH | 18) GATA3 | 19) PDS5B | 20) APOC3 |
| 21) STAG2 | 22) OAS1 | 23) OR13F1 | 24) OSR1 |
| 25) THBS3 | 26) APOB | 27) TTPA | 28) PDRG1 |
| 29) SULT1A1 | 30) OAS2 | 31) TAS2R43 | 32) APOA1 |
| 33) LRP6 | 34) GRIA3 | 35) F2RL3 | 36) KPNB1 |
| 37) IL10 | 38) RARA | 39) ART1 | 40) THBS1 |
| 41) CYP4A22 | 42) GRIA1 | 43) ALDH8A1 | 44) TLR4 |
| 45) COL9A1 | 46) IPO5 | 47) FBXO30 | 48) PICALM |
| 49) GP1BA | 50) RET | | |

A test was done on the 500 genes used to build the classifier to predict which genes are most informative to the classifier. This was done by measuring decrease in random forest classifier accuracy when the labels for that feature are shuffled. The top-ranked 50 most informative genes that were not already listed above are shown below:

| | | | |
|---|---|---|---|
| 1) LOC84931 | 2) DCC | 3) IFNG | 4) OXT |
| 5) CTAGE1 | 6) KCNA5 | 7) SPAG9 | 8) USP9X |
| 9) CRHBP | 10) PABPC1 | 11) SPG21 | 12) TTC17 |
| 13) ST6GALNAC6 | 14) S1PR2 | 15) MDGA2 | 16) CCR6 |
| 17) KCNJ14 | 18) KLRAP1 | 19) CTSH | 20) JMJD6 |
| 21) FOXS1 | 22) DICER1 | 23) HERC4 | 24) PDILT |
| 25) IKZF1 | 26) BLM | 27) FABP5 | 28) ACSL4 |
| 29) KIF2C | 30) SP1 | 31) IPO11 | 32) SLC38A2 |
| 33) MBP | 34) FOXE3 | 35) TET1 | 36) F3 |
| 37) ANKRD42 | 38) ULBP1 | 39) LPL | 40) ACP5 |
| 41) ADRA2B | | | |

1. A method to determine the probability that a test subject is afflicted with amyotrophic lateral sclerosis (ALS) which method comprises contacting motor neuron indicator cells with biological fluid of said test subject and comparing the expression pattern in said indicator cells to that obtained when said cells are contacted with biological fluid from normal subjects,
whereby an alteration in the expression pattern of the indicator cells contacted with the fluid from the test subject as compared to indicator cells contacted with fluid from normal subjects determines a high probability that a test subject is afflicted with ALS.

2. The method of claim 1 wherein said expression patterns are obtained by contacting mRNA extracted from said indicator cells or the corresponding cDNA with at least two probes complementary to an mRNA component of said cells or to its corresponding cDNA and detecting the binding of the probe to the mRNA or cDNA.

3. The method of claim 1 wherein said expression patterns comprise the level of expression of an exon of CK1γ2 that encodes the C-terminal palmitoylated region of said CK1γ2 whereby a diminished level of expression of this exon in cells contacted with fluid from the test subject as compared to its expression level in said indicator cells when contacted with biological fluid of normal subjects indicates a high probability that said test subject is afflicted with ALS.

4. The method of claim 3 wherein said exon encodes the human sequence SEQ ID NO:1, or the mouse sequence SEQ ID NO:3.

5. The method of claim 3 wherein the human exon has SEQ ID NO:2 and the mouse exon has SEQ ID NO:4.

6. The method of claim 3 wherein the at least one probe has the sequence SEQ ID NO:5 or its complement.

7. The method of claim 2 which comprises employing probes complementary to at least two mRNA or cDNA corresponding to genes selected from the group consisting of UBE2A, UBE2B, RNF8, UBR2, MARS, BCAR1, SPG21, SLA2, OAT, PYCR1, ALDH18A1, PYCR2, PYCRL, GARS, SMAD1, POLB, POLG2, TARS, TARS2, TARSL2, MTHFD1, MTHFD2, MTHFD1L, MTHFD2L, B4GALT1, B4GALT3, B4GALT2, WDFY3, SLC3A2, SLC8A2, SLC8A1, SLC8A3, INPP5A, INPP5B, INPP5J, INPP5K, NAT1, SLC1A4, SLC1A5, SLC38A3, SLC38A7, MTHFS, MTHFSD, MTHFR, SHMT1, SHMT2, FTCD, ALDH1L1, MTFMT, ALDH1L2, DHFR, GART, AMT, MTR, ATIC, TYMS, SLC36A4, SLC36A2, CLN8, GAA, GCH1, GLRA1, HEXA, SCN1A, TCF15, CNTNAP1, SLC7A1, SLC7A3, SLC7A5, SLC7A11, PIPDX, FGF2, SMAD3, SERPINE1, CASK, PTCH1, PTCH2, HHIP, GPT, GPT2, ASNS, ATF3, CCL2, CEBPZ, DDIT3, HERPUD1, IGFBP1, AARS, IARS, VARS, VARS2, LARS2, LARS, IARS2, IL18, PDE2A, PDE3A, VEGFA, FGFBP3, PGD, PHGDH, PSAT1, FOXC1, HEXB, CLN6, GPLD1, MEF2C, PPARGC1B, FGFR3, IHH, DDR2, TKT, FLT3, HELLS, HPRT, IMPDH1, IMPDH2, RAD23A, RAD23B, WNT10B, UBQLN4, DNASE1L1, DNASE1L2, DNASE1L3, TATDN2, TATDN3, ROS1, AGPAT9, PGK1, PGK2, FAS, FASN, NDUFAB1, HK1, KCNA4, KCNJ11, PKLR, PKM, PDXK, HDAC4, PHF2, KDM1A, KDM4C, PHF8, JHDM1D, EHMT2, SMYD2, EHMT1, SETD7, SETD3, CNN2, PRTN3, TGFB1, ADIPOQ, GNB2L1, EIF2AK3, HSPA5, EIF2A, EIF2S1, ATF4, DDR1, GLI2, LHX1, RELN, VLDLR, ARNT, EPAS1, HLF, HIF1A, HMOX1, SIN3A, FOXC2, PTGS2, HDAC7, SRPX2, ITPR1, ITPR2, ITPR3, CYTH3, BLM, MYC, TXNIP, NUMA1, PRM1, PRM2, ATXN7, SYNE1, HSF4, KDM3A, ABCA1, MTTP, ATG7, ATG10, PPP1R12A, SIP1, ZEB2, BMP2K, SBF2, PDK1, PDK2, PDK3, PDK4, BCKDK, KCNN1, KCNN2, KCNN3, KCNN4, EEF1E1, EPRS, QARS, AIMP2, AIMP1, RARS, DARS, KARS, NARS, CARS, HARS, FARSA, FARSB, PPA1, SARS, YARS, DHH, CSRP2BP, B4GALT4, ORC1, ORC2, SLC7A2, SLC25A15, SLC25A2, SNCA, MFN2, TIMM50, CDH1, FLNA, DDX58, EAF2, DMAP1, MAVS, TMEM173, CDK6, DRD1A, GFAP, GIF, LAMB2, MT3, POU3F2, EIF2B5, LAMC3, SUV39H1, BAZ2A, RRP8, SIRT1, FCER1G, HRG, SYK, TEC, GANC, MGA, MGAM, DECR1, ECSIT, MIOX, WDR93, CHRNA1, CHRND, VPS54, TSHZ3, DLAT, MLYCD, ACSS1, FGFR4, FIGF, CCL5, VEGFB, VEGFC, FBP1, PPARA, IER3, DDIT4, NCKAP1L, LCK, STAT5A, STAT5B, GIMAP5, CREBBP, TSC22D3, BHLHE40, STRA13, BHLHE41, SLC1A1, SLC1A2, SLC1A6, SLC1A7, TNFSF10, TNFRSF10B, FADD, CASP8, ACVR1, EFNA1, SOX4, TWIST1, IL2, IL21, GTPBP1, CARHSP1, EXOSC3, DIS3L, RS1, ARL6IP5, TRAT1, YRDC, PARP1, PNKP, MRPS35, MRPS26, MRPS11, MRPS9, SLC7A7, SLC7A15, SLC7A8, SLC7A4, SLC7A9, SLC7A10, SLC7A6, SLC7A6OS, SLC7A12, SLC7A13, SLC7A14, DNASE1, DNASE2A, SOX11, 5, NOTCH1, HDAC5, MYOCD, DNA2, MDP1, POLG, RNH1, DNAJA3, RRM2B, PEO1, RNASEH1, ENSA, KCNJ12, KCNMB2, KCNV1, PDZD3, TNFRSF11B, CALCA, CD38, INPP5D, P2RX7, TNFAIP3, CARTPT, KDR, PTPRJ, SDC4, SFRP1, TEK, TSC1, PPM1F, AMBP, BLVRA, BLVRB, HMOX2, SMAD4, TGFB2, NF1, POU3F1, SKI, ARHGEF10, ADAM22, LGI4, TOP1, TOP3A, TOP3B, TOP1MT, BMP4, FOXJ1, ZC3H8, NFKBID, BCKDHA, BCKDHB, DBT, NAT2, SAT1, LAT2, SLC43A1, SLC6A15, SLC38A1, SLC6A17, AGRP, CNR1, HTR1A, TACR3, QRFP, MIF, MC1R, AKAP5, AKAP12, CCR4, PARN, PAN2, CNOT6, CNOT6L, PIM1, LONP1, CLPX, CRBN, LONRF3, LONP2, LONRF1, LONRF2, ADM, HES1, RAMP2, HEY2, CCBL1, GLS, GLUD1, GLUL, GOT1, GOT2, PAH, GLS2, CAD, DFFA, DFFB and NME1, or the human orthologs thereof.

8. The method of claim 2 which comprises employing probes complementary to at least ten mRNA or cDNA corresponding to genes selected from the group consisting of UBE2A, UBE2B, RNF8, UBR2, MARS, BCAR1, SPG21, SLA2, OAT, PYCR1, ALDH18A1, PYCR2, PYCRL, GARS, SMAD1, POLB, POLG2, TARS, TARS2, TARSL2, MTHFD1, MTHFD2, MTHFD1L, MTHFD2L, B4GALT1, B4GALT3, B4GALT2, WDFY3, SLC3A2, SLC8A2, SLC8A1, SLC8A3, INPP5A, INPP5B, INPP5J, INPP5K, NAT1, SLC1A4, SLC1A5, SLC38A3, SLC38A7, MTHFS, MTHFSD, MTHFR, SHMT1, SHMT2, FTCD, ALDH1L1, MTFMT, ALDH1L2, DHFR, GART, AMT, MTR, ATIC, TYMS, SLC36A4, SLC36A2, CLN8, GAA, GCH1, GLRA1, HEXA, SCN1A, TCF15, CNTNAP1, SLC7A1, SLC7A3, SLC7A5, SLC7A11, PIPDX, FGF2, SMAD3, SERPINE1, CASK, PTCH1, PTCH2, HHIP, GPT, GPT2, ASNS, ATF3, CCL2, CEBPZ, DDIT3, HERPUD1, IGFBP1, AARS, JARS, VARS, VARS2, LARS2, LARS, IARS2, IL18, PDE2A, PDE3A, VEGFA, FGFBP3, PGD, PHGDH, PSAT1, FOXC1, HEXB, CLN6, GPLD1, MEF2C, PPARGC1B, FGFR3, IHH, DDR2, TKT, FLT3, HELLS, HPRT, IMPDH1, IMPDH2, RAD23A, RAD23B, WNT10B, UBQLN4, DNASE1L1, DNASE1L2, DNASE1L3, TATDN2, TATDN3, ROS1, AGPAT9, PGK1, PGK2, FAS, FASN, NDUFAB1, HK1, KCNA4, KCNJ11, PKLR, PKM, PDXK, HDAC4, PHF2, KDM1A, KDM4C, PHF8, JHDM1D, EHMT2, SMYD2, EHMT1, SETD7, SETD3, CNN2, PRTN3, TGFB1, ADIPOQ, GNB2L1, EIF2AK3, HSPA5, EIF2A, EIF2S1, ATF4, DDR1, GLI2, LHX1, RELN, VLDLR, ARNT, EPAS1, HLF, HIF1A, HMOX1, SIN3A, FOXC2, PTGS2, HDAC7, SRPX2, ITPR1, ITPR2, ITPR3, CYTH3, BLM, MYC, TXNIP, NUMA1, PRM1, PRM2, ATXN7, SYNE1, HSF4, KDM3A, ABCA1, MTTP, ATG7, ATG10, PPP1R12A, SIP1, ZEB2, BMP2K, SBF2, PDK1, PDK2, PDK3, PDK4, BCKDK, KCNN1, KCNN2, KCNN3, KCNN4, EEF1E1, EPRS, QARS, AIMP2, AIMP1, RARS, DARS, KARS, NARS, CARS, HARS, FARSA, FARSB, PPA1, SARS, YARS, DHH, CSRP2BP, B4GALT4, ORC1, ORC2, SLC7A2, SLC25A15, SLC25A2, SNCA, MFN2, TIMM50, CDH1, FLNA, DDX58, EAF2, DMAP1, MAVS, TMEM173, CDK6, DRD1A, GFAP, GIF, LAMB2, MT3, POU3F2, EIF2B5, LAMC3, SUV39H1, BAZ2A, RRP8, SIRT1, FCER1G, HRG, SYK, TEC, GANC, MGA, MGAM, DECR1, ECSIT, MIOX, WDR93, CHRNA1, CHRND, VPS54, TSHZ3, DLAT, MLYCD, ACSS1, FGFR4, FIGF, CCL5, VEGFB, VEGFC, FBP1, PPARA, IER3, DDIT4, NCKAP1L, LCK, STAT5A, STAT5B, GIMAP5, CREBBP, TSC22D3, BHLHE40, STRA13, BHLHE41, SLC1A1, SLC1A2, SLC1A6, SLC1A7, TNFSF10, TNFRSF10B, FADD, CASP8, ACVR1, EFNA1, SOX4, TWIST1, IL2, IL21, GTPBP1, CARHSP1, EXOSC3, DIS3L, RS1, ARL6IP5, TRAT1, YRDC, PARP1, PNKP, MRPS35, MRPS26, MRPS11, MRPS9, SLC7A7, SLC7A15, SLC7A8, SLC7A4, SLC7A9, SLC7A10, SLC7A6, SLC7A6OS, SLC7A12, SLC7A13, SLC7A14, DNASE1, DNASE2A, SOX11, 5, NOTCH1, HDAC5, MYOCD, DNA2, MDP1, POLG, RNH1, DNAJA3, RRM2B, PEO1, RNASEH1, ENSA, KCNJ12, KCNMB2, KCNV1, PDZD3, TNFRSF11B, CALCA, CD38, INPP5D, P2RX7, TNFAIP3, CARTPT, KDR, PTPRJ, SDC4, SFRP1, TEK, TSC1, PPM1F, AMBP, BLVRA, BLVRB, HMOX2, SMAD4, TGFB2, NF1, POU3F1, SKI, ARHGEF10, ADAM22, LGI4, TOP1, TOP3A, TOP3B, TOP1MT, BMP4, FOXJ1, ZC3H8, NFKBID, BCKDHA, BCKDHB, DBT, NAT2, SAT1, LAT2, SLC43A1, SLC6A15, SLC38A1, SLC6A17, AGRP, CNR1, HTR1A, TACR3, QRFP, MIF, MC1R, AKAP5, AKAP12, CCR4, PARN, PAN2, CNOT6, CNOT6L, PIM1, LONP1, CLPX, CRBN, LONRF3, LONP2, LONRF1, LONRF2, ADM, HES1, RAMP2, HEY2, CCBL1, GLS, GLUD1, GLUL, GOT1, GOT2, PAH, GLS2, CAD, DFFA, DFFB and NME1, or the human orthologs thereof.

9. The method of claim 2 which comprises employing probes complementary to at least fifty mRNA or cDNA corresponding to genes selected from the group consisting of UBE2A, UBE2B, RNF8, UBR2, MARS, BCAR1, SPG21, SLA2, OAT, PYCR1, ALDH18A1, PYCR2, PYCRL, GARS, SMAD1, POLB, POLG2, TARS, TARS2, TARSL2, MTHFD1, MTHFD2, MTHFD1L, MTHFD2L, B4GALT1, B4GALT3, B4GALT2, WDFY3, SLC3A2, SLC8A2, SLC8A1, SLC8A3, INPP5A, INPP5B, INPP5J, INPP5K, NAT1, SLC1A4, SLC1A5, SLC38A3, SLC38A7, MTHFS, MTHFSD, MTHFR, SHMT1, SHMT2, FTCD, ALDH1L1, MTFMT, ALDH1L2, DHFR, GART, AMT, MTR, ATIC, TYMS, SLC36A4, SLC36A2, CLN8, GAA, GCH1, GLRA1, HEXA, SCN1A, TCF15, CNTNAP1, SLC7A1, SLC7A3, SLC7A5, SLC7A11, PIPDX, FGF2, SMAD3, SERPINE1, CASK, PTCH1, PTCH2, HHIP, GPT, GPT2, ASNS, ATF3, CCL2, CEBPZ, DDIT3, HERPUD1, IGFBP1, AARS, IARS, VARS, VARS2, LARS2, LARS, IARS2, IL18, PDE2A, PDE3A, VEGFA, FGFBP3, PGD, PHGDH, PSAT1, FOXC1, HEXB, CLN6, GPLD1, MEF2C, PPARGC1B, FGFR3, IHH, DDR2, TKT, FLT3, HELLS, HPRT, IMPDH1, IMPDH2, RAD23A, RAD23B, WNT10B, UBQLN4, DNASE1L1, DNASE1L2, DNASE1L3, TATDN2, TATDN3, ROS1, AGPAT9, PGK1, PGK2, FAS, FASN, NDUFAB1, HK1, KCNA4, KCNJ11, PKLR, PKM, PDXK, HDAC4, PHF2, KDM1A, KDM4C, PHF8, JHDM1D, EHMT2, SMYD2, EHMT1, SETD7, SETD3, CNN2, PRTN3, TGFB1, ADIPOQ, GNB2L1, EIF2AK3, HSPA5, EIF2A, EIF2S1, ATF4, DDR1, GLI2, LHX1, RELN, VLDLR, ARNT, EPAS1, HLF, HIF1A, HMOX1, SIN3A, FOXC2, PTGS2, HDAC7, SRPX2, ITPR1, ITPR2, ITPR3, CYTH3, BLM, MYC, TXNIP, NUMA1, PRM1, PRM2, ATXN7, SYNE1, HSF4, KDM3A, ABCA1, MTTP, ATG7, ATG10, PPP1R12A, SIP1, ZEB2, BMP2K, SBF2, PDK1, PDK2, PDK3, PDK4, BCKDK, KCNN1, KCNN2, KCNN3, KCNN4, EEF1E1, EPRS, QARS, AIMP2, AIMP1, RARS, DARS, KARS, NARS, CARS, HARS, FARSA, FARSB, PPA1, SARS, YARS, DHH, CSRP2BP, B4GALT4, ORC1, ORC2, SLC7A2, SLC25A15, SLC25A2, SNCA, MFN2, TIMM50, CDH1, FLNA, DDX58, EAF2, DMAP1, MAVS, TMEM173, CDK6, DRD1A, GFAP, GIF, LAMB2, MT3, POU3F2, EIF2B5, LAMC3, SUV39H1, BAZ2A, RRP8, SIRT1, FCER1G, HRG, SYK, TEC, GANC, MGA, MGAM, DECR1, ECSIT, MIOX, WDR93, CHRNA1, CHRND, VPS54, TSHZ3, DLAT, MLYCD, ACSS1, FGFR4, FIGF, CCL5, VEGFB, VEGFC, FBP1, PPARA, IER3, DDIT4, NCKAP1L, LCK, STAT5A, STAT5B, GIMAP5, CREBBP, TSC22D3, BHLHE40, STRA13, BHLHE41, SLC1A1, SLC1A2, SLC1A6, SLC1A7, TNFSF10, TNFRSF10B, FADD, CASP8, ACVR1, EFNA1, SOX4, TWIST1, IL2, IL21, GTPBP1, CARHSP1, EXOSC3, DIS3L, RS1, ARL6IP5, TRAT1, YRDC, PARP1, PNKP, MRPS35, MRPS26, MRPS11, MRPS9, SLC7A7, SLC7A15, SLC7A8, SLC7A4, SLC7A9, SLC7A10, SLC7A6, SLC7A6OS, SLC7A12, SLC7A13, SLC7A14, DNASE1, DNASE2A, SOX11, 5, NOTCH1, HDAC5, MYOCD, DNA2, MDP1, POLG, RNH1, DNAJA3, RRM2B, PEO1, RNASEH1, ENSA, KCNJ12, KCNMB2, KCNV1, PDZD3, TNFRSF11B, CALCA, CD38, INPP5D, P2RX7, TNFAIP3, CARTPT, KDR, PTPRJ, SDC4, SFRP1, TEK, TSC1, PPM1F, AMBP, BLVRA, BLVRB, HMOX2, SMAD4, TGFB2, NF1, POU3F1, SKI, ARHGEF10, ADAM22, LGI4, TOP1, TOP3A, TOP3B, TOP1MT, BMP4, FOXJ1, ZC3H8, NFKBID, BCKDHA, BCKDHB, DBT, NAT2, SAT1, LAT2, SLC43A1, SLC6A15, SLC38A1, SLC6A17, AGRP, CNR1, HTR1A, TACR3, QRFP, MIF, MC1R, AKAP5, AKAP12, CCR4, PARN, PAN2, CNOT6, CNOT6L, PIM1, LONP1, CLPX, CRBN, LONRF3, LONP2, LONRF1, LONRF2, ADM, HES1, RAMP2, HEY2, CCBL1, GLS, GLUD1, GLUL, GOT1, GOT2, PAH, GLS2, CAD, DFFA, DFFB and NME1, or the human orthologs thereof.

10. The method of claim 7 wherein said genes are selected from the same gene set.

11. The method of claim 2 which comprises employing probes complementary to at mRNA or cDNA corresponding to the transcription factors ATF4 and/or CHOP and/or their targets.

12. The method of claim 1 wherein the biological fluid is serum or cerebrospinal fluid (CSF).

13. The method of claim 1 wherein the test subjects and normal subjects are human.

14. A method to determine the probability of the presence of presymptomatic or symptomatic Alzheimer's disease (PSAD) in a test subject which method comprises using an indicator cell assay (iCAP) by contacting indicator cells that are pan neuronal populations of glutamatergic (and/or GABAergic) neurons with biological fluid of said test subject and comparing the expression pattern in said indicator cells to that obtained when said cells are contacted with biological fluid from normal subjects,
whereby an alteration in the expression pattern of the indicator cells contacted with the fluid from the test subject as compared to indicator cells contacted with fluid from normal subjects determines a high probability that a test subject is presymptomatic for AD.

15. The method of claim 14 wherein said expression patterns are obtained by contacting mRNA extracted from said indicator cells or the corresponding cDNA with at least two probes complementary to an mRNA or cDNA component of said cells and detecting the binding of the probes to the mRNA or cDNA.

16. The method of claim 15 which comprises employing probes complementary to at least two mRNA or cDNA corresponding to genes selected from the group consisting of MYLK2, TOMM20L, APOE, ZNF675, MYLK3, SULT2B1, GRIA2, LCAT, GRIA4, IL18, OSR2, ZNF525, IL4, TAS2R50, GHRL, DBP, IHH, GATA3, PDS5B, APOC3, STAG2, OAS1, OR13F1, OSR1, THBS3, APOB, TTPA, PDRG1, SULT1A1, OAS2, TAS2R43, APOA1, LRP6, GRIA3, F2RL3, KPNB1, IL10, RARA, ART1, THBS1, CYP4A22, GRIA1, ALDH8A1, TLR4, COL9A1, IPO5, FBXO30, PICALM, GP1BA and RET and/or the group consisting of LOC84931, DCC, IFNG, OXT, CTAGE1, KCNA5, SPAG9, USP9X, CRHBP, PABPC1, SPG21, TTC17, ST6GALNAC6, S1PR2, MDGA2, CCR6, KCNJ14, KLRAP1, CTSH, JMJD6, FOXS1, DICER1, HERC4, PDILT, IKZF1, BLM, FABP5, ACSL4, KIF2C, SP1, IPO11, SLC38A2, MBP, FOXE3, TET1, F3, ANKRD42, ULBP1, LPL, ACP5 and ADRA2B.

17. The method of claim 15 which comprises employing probes complementary to at least ten mRNA or cDNA corresponding to genes selected from the group consisting of MYLK2, TOMM20L, APOE, ZNF675, MYLK3, SULT2B1, GRIA2, LCAT, GRIA4, IL18, OSR2, ZNF525, IL4, TAS2R50, GHRL, DBP, IHH, GATA3, PDS5B, APOC3, STAG2, OAS1, OR13F1, OSR1, THBS3, APOB, TTPA, PDRG1, SULT1A1, OAS2, TAS2R43, APOA1, LRP6, GRIA3, F2RL3, KPNB1, IL10, RARA, ART1, THBS1, CYP4A22, GRIA1, ALDH8A1, TLR4, COL9A1, IPO5, FBXO30, PICALM, GP1BA and RET and/or the group consisting of LOC84931, DCC, IFNG, OXT, CTAGE1, KCNA5, SPAG9, USP9X, CRHBP, PABPC1, SPG21, TTC17, ST6GALNAC6, S1PR2, MDGA2, CCR6, KCNJ14, KLRAP1, CTSH, JMJD6, FOXS1, DICER1, HERC4, PDILT, IKZF1, BLM, FABP5, ACSL4, KIF2C, SP1, IPO11, SLC38A2, MBP, FOXE3, TET1, F3, ANKRD42, ULBP1, LPL, ACP5 and ADRA2B.

18. The method of claim 15 which comprises employing probes complementary to at least fifty mRNA or cDNA corresponding to genes selected from the group consisting of MYLK2, TOMM20L, APOE, ZNF675, MYLK3, SULT2B1, GRIA2, LCAT, GRIA4, IL18, OSR2, ZNF525, IL4, TAS2R50, GHRL, DBP, IHH, GATA3, PDS5B, APOC3, STAG2, OAS1, OR13F1, OSR1, THBS3, APOB, TTPA, PDRG1, SULT1A1, OAS2, TAS2R43, APOA1, LRP6, GRIA3, F2RL3, KPNB1, IL10, RARA, ART1, THBS1, CYP4A22, GRIA1, ALDH8A1, TLR4, COL9A1, IPO5, FBXO30, PICALM, GP1BA and RET and/or the group consisting of LOC84931, DCC, IFNG, OXT, CTAGE1, KCNA5, SPAG9, USP9X, CRHBP, PABPC1, SPG21, TTC17, ST6GALNAC6, S1PR2, MDGA2, CCR6, KCNJ14, KLRAP1, CTSH, JMJD6, FOXS1, DICER1, HERC4, PDILT, IKZF1, BLM, FABP5, ACSL4, KIF2C, SP1, IPO11, SLC38A2, MBP, FOXE3, TET1, F3, ANKRD42, ULBP1, LPL, ACP5 and ADRA2B.

19. The method of claim 16 wherein said genes are selected from the same gene set.

20. The method of claim 14 wherein the biological fluid is serum or cerebrospinal fluid (CSF).

21. The method of claim 14 wherein the test subjects and normal subjects are human.


SEQUENCE LISTING (6 sequences)

SEQ ID NO: 1 - protein, 18 aa, Homo sapiens
KCCCFFKRRKRKSLQRHK

SEQ ID NO: 2 - DNA, 57 bp, Homo sapiens
aaatgctgctgtttcttcaagaggagaaagagaaaatcgctgcagcgacacaagtga

SEQ ID NO: 3 - protein, 18 aa, Mus musculus
KCCCFFKRRKRKSLQRHK

SEQ ID NO: 4 - DNA, 57 bp, Mus musculus
aagtgctgctgcttcttcaagaggagaaagagaaaatcgctgcagcgacataagtga

SEQ ID NO: 5 - DNA, 21 bp, Artificial Sequence
  Synthetic Construct
aaatcgctgcagcgacataag

SEQ ID NO: 6 - protein, 442 aa, Mus musculus
MDFDKKGGKGELEEGRRMSKTGTSRSNHGVRSSGTSSGVLMVGPNFRVGKKIGCGNFGELRLGKNLYTNEYVAIKLEPIKSRAPQLHLEYRFYKQLSTTGEADSGTGPALLGQQWLRTPSMDVSFAEGVPQVYYFGPCGKYNAMVLELLGPSLEDLFDLCDRTFTLKTVLMIAIQLITRMEYVHTKSLIYRDVKPENFLVGRPGSKRQHSIHIIDFGLAKEYIDPETKKHIPYREHKSLTGTARYMSINTHLGKEQSRRDDLEALGHMFMYFLRGSLPWQGLKADTLKERYQKIGDTKRATPIEVLCESFPEEMATYLRYVRRLDFFEKPDYDYLRKLFTDLFDRSGYVFDYEYDWAGKPLPTPIGTVHPDVPSQPPHRDKAQLHTKNQALNSTNGELNTDDPTAGHSNAPIAAPAEVEVADETKCCCFFKRRKRKSLQRHK